(19) United States
(12) Patent Application Publication
(10) Pub. No.: US 2026/0033954 A1
(43) Pub. Date: Feb. 5, 2026

(54) Methods and Apparatuses for Prevention and Treatment of Scapular Spine Fractures

(71) Applicants: T Bradley EDWARDS, Houston, TX (US); Mark R. BRINKER, Houston, TX (US)

(72) Inventors: T Bradley EDWARDS, Houston, TX (US); Mark R. BRINKER, Houston, TX (US)

(21) Appl. No.: 18/929,536

(22) Filed: Oct. 28, 2024

Related U.S. Application Data
(60) Provisional application No. 63/678,294, filed on Aug. 1, 2024.
(60) Provisional application No. 63/681,126, filed on Aug. 8, 2024.
(60) Provisional application No. 63/683,386, filed on Aug. 15, 2024.

(51) Int. Cl. A61F 2/40

(52) U.S. Cl. CPC A61F 2/4081; A61F 2002/4085

(57) ABSTRACT

An elongated implant for prophylactic treatment and post-fracture treatment of fractures of the spina scapulae incident to reverse total should arthroplasty, the elongated implant having an intermediate portion between two end portions, which intermediate portion may span all or a portion of the Levy zone II fracture area.

### CROSS REFERENCE TO RELATED APPLICATIONS

This application claims benefit of and priority to U.S. Provisional Application No. 63/678,294, filed on Aug. 1, 2024, and U.S. Provisional Application No. 63/681,126, filed on Aug. 8, 2024, and U.S. Provisional Application No. 63/683,386, filed on Aug. 15, 2024, the entire contents of each of which are incorporated herein for all purposes.

### STATEMENT REGARDING FEDERALLY SPONSORED RESEARCH OR DEVELOPMENT

Not applicable.

### REFERENCE TO APPENDIX

Not applicable.

### BACKGROUND OF THE INVENTION

Field of the Invention The present inventions relate to implants and/or prostheses for strengthening the scapular spine against fracture, and to implants and/or prostheses for repairing a fracture of the scapular spine.

Description of the Related Art Conventional total shoulder arthroplasty (TSA) involves replacing the ball on the end of the humerus with a ball prosthesis and replacing 20) the socket in the scapula with a socket prosthesis. Today, however, there are more reverse total shoulder arthroplasties performed in the US than conventional TSAs. In reverse total shoulder arthroplasty (rTSA) the metal ball is attached to the scapula, and the socket is attached to the upper end of the humerus.

For reasons that may not be completely understood, there have been fractures of the scapular spine (spina scapulae) after recovering from an rTSA. Acromion stress fractures and scapular spine fractures occur in about 10% of rTSAs. These fractures are more often present as Levy II fractures, but also as Levy I and Levy III fractures. See Predictive Factors of Acromial Fractures Following Reverse Total Shoulder Arthroplasty: A Subgroup Analysis of 860 Shoulders, JSES Int., September 2023. It appears that these fractures after rTSA may be caused by a downward bending force or moment applied to the spina scapulae. It is also thought that these fractures, or at least pre-fractures, may be caused by traction during an rTSA procedure

To date, these types of scapular spine fractures have been treated by surgical and non-surgical procedures with limited success. Surgical treatments include open reduction implantation of locking or non-locking plate(s) secured to the backside of spina scapulae, below the spina scapulae, or double plating the spina scapulae through open reduction internal fixation.

For example, U.S. Pat. No. 12,016,601 is entitled Acromion Fracture Repair System and discloses “[a] kit including (1) a plate configured to be secured to a scapular spine with a first end of the plate near a trigonum and a second end of the plate near an acromion; (2) a first hook including a mount, a first hook portion extending from the mount in a first direction, a spacer extending from the first hook portion in a transverse direction, and a second hook portion extending from an opposite end of the spacer in the first direction, the first hook adapted to extend around a lateral end of the acromion when fixed to the second end of the plate; and (3) a second hook including a mount, a curved portion curving away from the mount, and a hook portion at an opposite end of the curved portion, the second hook adapted to extend around the trigonum when fixed to the first end of the plate.”

PCT International Publication No. WO 2023/203042 is entitled Medical Implant for Treatment of a Scapular Fracture and discloses “[a] medical implant (100) for treatment of one or more fractures of a scapula (200) of a subject, comprises: a longitudinal body (18) having a medial end (10) and a lateral end (12), a cranial side (14), and an caudal side (16); a scapular spine portion (SSP) (20) of the longitudinal body (18), located at the medial end (10), having an SSP co-operating surface (22) configured for co-operation with a scapular spine (220) of the subject; an acromion portion (AP) (60) of the longitudinal body (18), located at the lateral end (16), having an AP co-operating surface (62) configured for co-operation with a caudal surface (212) of the acromion (210) of the subject; and bridging portion (40) of the longitudinal body (18) connecting the scapular spine portion (SSP) (20) to the acromion portion (AP) (60), and configured to maintain the scapular spine portion (SSP) (20) in fixed positional relation with the acromion portion (AP) (60); wherein the SSP (20) co-operating surface (22) is deposed on the caudal side (16) and the AP (60) co-operating surface (62) is disposed on the cranial side (14) of the implant (100).”

Thus, there is a need for an internal fixation fracture treatment that does not require open reduction, and there is a need for a minimally invasive prophylactic treatment to prevent fractures from occurring post-rTSA.

It is to be understood that the discussion above is provided for illustrative purposes only and is not intended to and does not limit the scope or subject matter of the appended or ultimately issued claims or those of any related patent application or patent. Thus, none of the appended claims, ultimately issued claims or claims of any related application or patent are to be limited by the above discussion or construed to address, include, or exclude each or any of the above-cited features or disadvantages merely because such were mentioned herein.

### BRIEF SUMMARY OF THE INVENTION

A brief summary of the inventions indicating their nature and substance may be understood from the subject matter presented in the claims filed with this application, which are incorporated into this brief summary by reference for all purposes, and by the inventions presented in any claims that may be issued from this application, which claims also are incorporated by reference into this brief summary by reference for all purposes.

### BRIEF DESCRIPTION OF THE SEVERAL VIEWS OF THE DRAWINGS

The following figures form part of the disclosure of our inventions and are included to demonstrate certain further aspects of the inventions. The inventions may be better understood by reference to one or more of these figures in combination with the detailed description of certain embodiments presented herein.

FIG. 1 illustrates a posterior view of a human skeleton showing the right scapula and scapular spine.

FIG. 2 illustrates the skeleton of FIG. 1, but with a reverse total shoulder prosthesis.

FIGS. 3A, 3B and 3C illustrate an embodiment of a scapular spine implant.

FIG. 4 illustrates additional embodiments of a scapular spine implant.

FIG. 5 illustrates a scapular spine implant according to the present inventions in position relative to a scapular spine.

.

FIG. 6 illustrates another embodiment of a scapular spine implant having an integral targeting device component.

FIG. 7 illustrates the implant of FIG. 7 positioned in the targeting device receptacle.

FIG. 8 illustrates the external targeting device with a scapular spine implant.

FIG. 9 illustrates a posterior view of a body with a right shoulder RT, a lateral incision for implantation of a scapular spine implant and an optional medial incision.

FIG. 10 illustrates implantation of a scapular spine implant.

FIG. 11 illustrates the scapular spine implant in final position relative to the scapular spine.

FIG. 12 illustrates the scapular spine implant secured to the scapular spine.

While the inventions disclosed herein are susceptible to various modifications and alternative forms, only a few specific embodiments have been shown by way of example in the drawings and are described in more detail below. The figures and detailed descriptions of these embodiments are not intended to limit the breadth or scope of the inventive concepts or the appended claims in any manner. Rather, the figures and detailed written descriptions are provided to illustrate the inventive concepts to a person of ordinary skill in the art and to enable such person to make and use the inventive concepts illustrated and taught by the specific embodiments.

### DETAILED DESCRIPTION

The written description of specific structures and functions below, are not presented to limit the scope of the inventions disclosed or the scope of the appended claims. Rather, the Figures and written description are provided to instruct a person skilled in this art to make and use the inventions for which patent protection is sought.

A person of skill in this art that has benefit of this disclosure will understand that the inventions are disclosed and taught herein by reference to specific embodiments, and that these specific embodiments are susceptible to numerous and various modifications and alternative forms without departing from the inventions we possess. For example, and not limitation, a person of skill in this art that has benefit of this disclosure will understand that Figures and/or embodiments that use one or more common structures or elements, such as a structure or an element identified by a common reference number, are linked together for all purposes of supporting and enabling our inventions, and that such individual Figures or embodiments are not disparate disclosures. A person of skill in this art that has benefit of this disclosure immediately will recognize and understand the various other embodiments of our inventions having one or more of the structures or elements illustrated and/or described in the various linked embodiments. In other words, not all possible embodiments of our inventions are described or illustrated in this application, and one or more of the claims to our inventions may not be directed to a specific, disclosed example. Nonetheless, a person of skill in this art that has benefit of this disclosure will understand that the claims are fully supported by the entirety of this disclosure.

People skilled in this art will appreciate that not all features of a commercial embodiment of the inventions are described or shown for the sake of clarity and understanding. Persons of skill in this art will also appreciate that the development of an actual commercial embodiment incorporating aspects of the present inventions will require numerous implementation-specific decisions to achieve the developer's ultimate goal for the commercial embodiment. Such implementation-specific decisions may include, and likely are not limited to, compliance with system-related, business-related, government-related, and other constraints, which may vary by specific implementation, location and from time to time. While a developer's efforts might be complex and time-consuming in an absolute sense, such efforts would be, nevertheless, a routine undertaking for those of skill in this art that have benefit of this disclosure.

Further, the use of a singular term, such as, but not limited to, “a,” is not intended as limiting of the number of items. Also, the use of relational terms, such as, but not limited to, “top,” “bottom,” “left,” “right,” “upper,” “lower,” “down,” “up,” “side,” and the like are used in the written description for clarity in specific reference to the Figures and are not intended to limit the scope of the invention or the scope of what is claimed.

The term “about” or “approximately” as used herein to modify a measurable value is meant to encompass variations of +/−10% or less, and preferably +/−5% or less, and still more preferably +/−0.1% or less of and from the specified value. It is to be understood that the value to which the modifier “about” or “approximately” refers is itself also specifically, and preferably, disclosed without modification.

Reference throughout this disclosure to “one embodiment,” “an embodiment,” “some embodiments” or similar language means that a particular feature, structure, or characteristic described in connection with the embodiment is included in at least one of the many possible embodiments of the present inventions. The terms “including,” “comprising,” “having,” and variations thereof mean “including but not limited to” unless expressly specified otherwise. An enumerated listing of items does not imply that any or all the items are mutually exclusive and/or mutually inclusive, unless expressly specified otherwise. The terms “a,” “an,” and “the” also refer to “one or more” unless expressly specified otherwise.

Furthermore, the described features, structures, or characteristics of one embodiment may be combined in any suitable manner in one or more other embodiments. Those of skill in the art having the benefit of this disclosure will understand that the inventions may be practiced without one or more of the specific details, or with other methods, components, materials, and so forth. In other instances, well-known structures, materials, or operations are not shown or described in detail to avoid obscuring aspects of the disclosure.

The description of elements in each Figure may refer to elements of proceeding Figures. Like numbers refer to like elements in all figures, including alternate embodiments of like elements. In some embodiments, the functions/actions/structures noted in the figures may occur out of the order noted in the block diagrams and/or operational illustrations. For example, two operations shown as occurring in succession, in fact, may be executed substantially concurrently or the operations may be executed in the reverse order, depending upon the functionality/acts/structure involved.

Our inventions comprise surgical methods and implants for repairing an existing fracture of the spina scapulae and surgical methods and implants for the prevention of spina scapulae fractures, particularly fractures after an RT. For the latter, it is preferred, but not required, that the preventative treatment be performed before, during and/or as part of the RT. Our inventions further comprise a minimally invasive percutaneous technique in which a first, single incision is made at a lateral position along the scapula spine or acromion and the implant is inserted or guided along the edge of the spina scapulae in a medial direction. Optionally, a second percutaneous incision may be made at a medial location along the spina scapulae to correctly locate the medial end of the implant.

Implants useful with our inventions may comprise an elongated plate sized and shaped to be attached to, preferably, the posterior edge of the scapular spine spanning, preferably, at least a portion of the Levy II fracture position. The plate may utilize locking screws or non-locking screws or a combination of both, but a locking screw type plate is presently preferred. The plate may comprise, and preferably does comprise, a reference surface disposed normal to the long axis of the implant that is sized and shaped to contact at least a portion of the rostral or superior surface of the scapular spine, preferably, but not exclusively, at a medial location.

It will be understood that the implant may be inserted along the spine edge through the lateral incision such that the reference surface contacts the upper surface of the spine at a medial location. Once positioned, the lateral end of the implant may be rotated about the contact between the reference surface and spine in a rostral or caudal direction to correctly locate the implant along the spine edge without disturbing placement of the medial end of the implant. Additionally, or optionally, a second medial incision may be made to expose the medial end of the implant for accurate location on the spine and/or for fixation. For example, if a second incision is made, a temporary locating pin may be inserted through a hole adjacent to the medial end of the implant while the lateral end is located and fixed to the spina scapulae. The optional second incision may be employed with implants that have or do not have a medial reference surface.

In a preferred embodiment, an external targeting device is used along with the implant to aid insertion of the implant through the first incision, to locate the implant along the spine edge and/or to guide the placement of locking and/or non-locking screws. For these embodiments, a lateral portion of the implant may comprise a targeting device component, such as, but not limited to, a threaded receptacle.

In use, it is contemplated that during an rTSA procedure, such as before beginning the rTSA procedure, the implant of our inventions may be implanted and fixed to the spine. For example, the implant may be removably secured to the external targeting guide, the first lateral incision made, the implant/guide assembly manipulated by hand to insert the medial end of the implant through the first incision and guide the implant along the spine edge so that the reference surface, if provided, contacts an upper or superior surface of the spine. The lateral portion of the implant may then be positionally adjusted so that the entire implant is correctly located along the spine edge. The holes in the guide are then used to locate and install the screws to fix the implant to the spina scapulae, after which the guide can be removed from the lateral end of the implant and the incision closed.

While the former procedure was described in the context of a pre-fracture or prophylactic minimally invasive operation, it will be appreciated that a post-fracture procedure may be performed substantially identically. In both types of procedures, it is preferred that the screws be placed to avoid the common areas of fracture that have been observed, such as Levy position II.

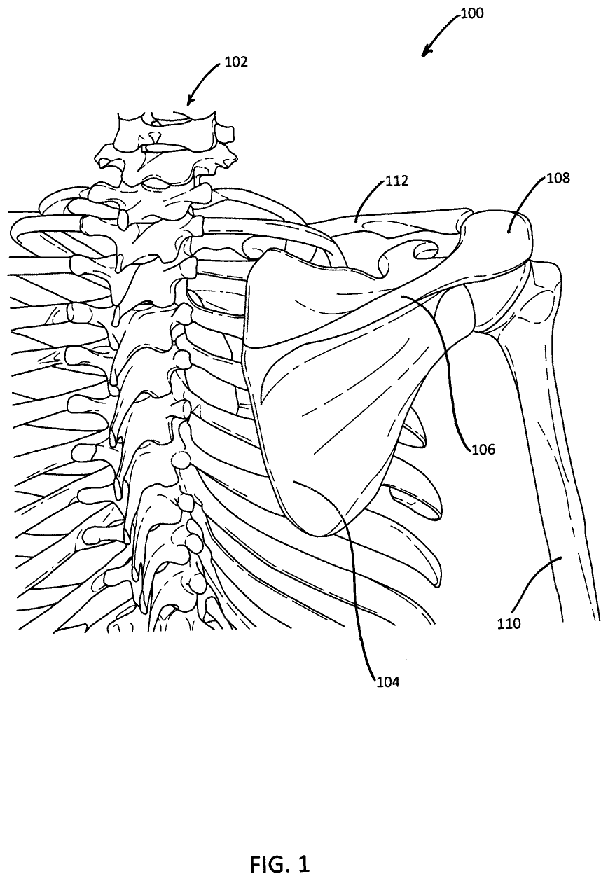

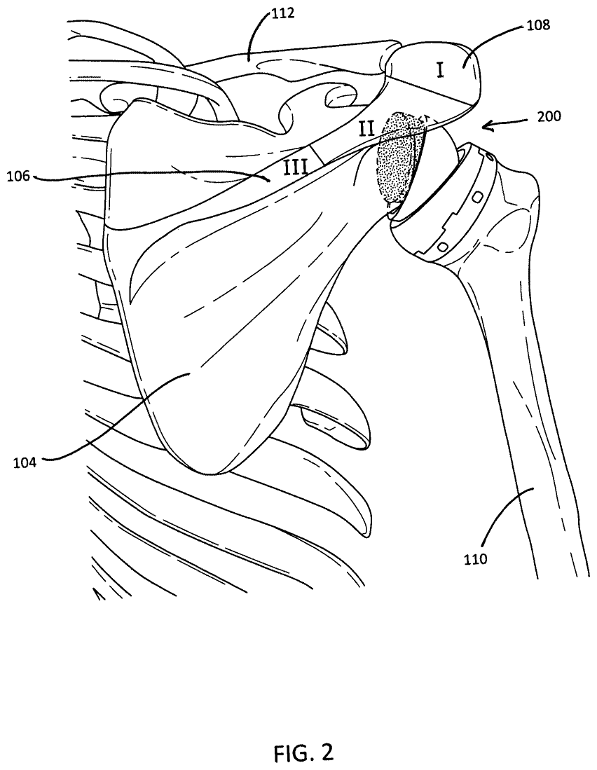

Turning to the figures, FIG. 1 illustrates a posterior view of a human skeleton 100 showing the vertebral column 102, the right scapula 104, with scapular spine 106 and acromion 108, humerus 110, and clavicle 112. FIG. 2 is a closer view of the right scapula 104 after a reverse total shoulder prosthesis 200 has been implanted. The scapular spine 106 is shown with the Levy fracture zones I, II, and III illustrated. As discussed previously, fractures of the scapular spine have been reported post-rTSA primarily in Levy zone II.

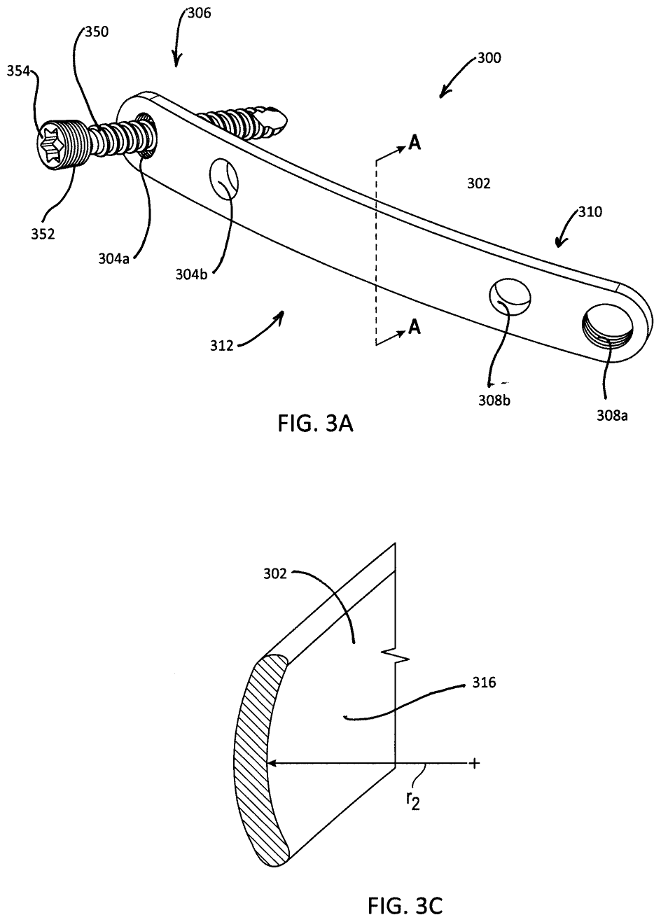

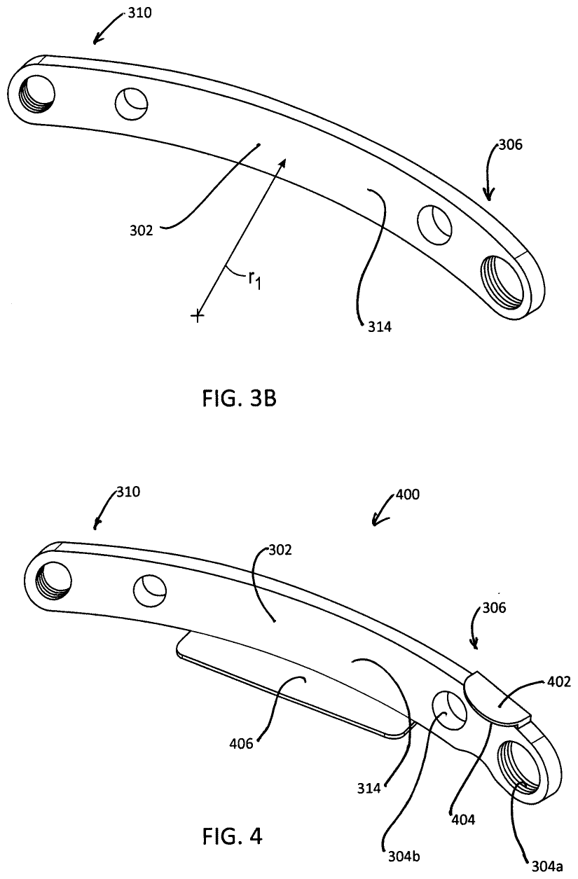

FIG. 3A illustrates a plate-like implant 300 comprising an elongated body 302. The implant 300 has an axial length, L, that is, preferably, long enough to span all or a portion of Levy zone II. For example, it is preferred that the length, L, is sufficient to extend from a location in Levy zone III to a location in Levy zone I. In practical terms, length, L, may range from about 5 cm to about 12 cm, and preferably is about 10 cm in length. The implant 300 is shown to have two screw head sockets 304a and 304b in a first end portion 306 and two screw head sockets 308a and 308b in a second end portion 310. Sockets 304a and 308a are illustrated as threaded sockets for locking bone screws, such as self-tapping, locking screw 350. Sockets 304b and 308b are illustrated as non-threaded sockets for non-locking bone screws (not shown). It will be understood that a non-locking screw does not have threads, such as tapered threads 352, on the screw head 354, which provide locking engagement with threads in the socket.

It is preferred that the implant 300 is implanted on the spine 106 such that the portion 312 intermediate the end portions spans at least a portion of, and preferably all, the Levy Zone II. Thus, implant 300 is illustrated not to have screw sockets in the intermediate portion 312 between the first end portion 306 and the second end portion 310 so that screws are not inserted in zone II. Because post-rTSA scapular spine fractures may occur more often in zone II, it is preferred not to create a stress riser or structural discontinuity in zone II for prophylactic (i.e., pre-fracture) implants. While it is preferred not to implant screws in the intermediate portion 312, our inventions contemplate that one or more screw head sockets may be located in the intermediate portion 312 and one or more screws implanted in that portion. For example, and not limitation, if the implant 300 is used to treat a fracture in zone II, rather than to prophylactically reduce or prevent fractures, it may be desirable to implant bone screws in zone II on either side of the fracture to provide additional fixation.

The implant 300 may be flat or planar, or as illustrated in FIG. 3B, implant 300 may have a concave or contoured surface 314 along the axial length, L, shown by radius r₁. If an axial concave surface 314 is utilized, it is preferred that the concave surface 314 face or contact the scapular spine 106. As illustrated in FIG. 3C, implant 300 may have a concave or contoured surface 316 transverse to the axial length, L, shown by radius r₂. If a transverse concave surface 316 is utilized, it is preferred that the concave surface 316 face or contact the scapular spine 106. It will be understood that embodiments of implant 300 may be flat, may have an axial concave surface, may have a transverse concave surface, or may have both an axial and transverse concave surface. Regardless of whether the implant utilizes one or more concave surfaces, the outer surface of implant may be flat or planar, or curved.

The implant 300 may be manufactured from one more biocompatible material, including, stainless steel, titanium and its alloys, Co—Cr, carbon fiber reinforced composites, ceramic, PMMA bone cement, pyrocarbon, and/or bone graft. The surface of the implant that contacts the scapular spine may include surface coatings or surface textures to provide enhance fixation and/or. Any of the exemplary embodiments described herein can be fabricated by traditional computer aided manufacturing processes, forged, cast, injected molded, or by using additive manufacturing or similar processes. It is preferred that the implant 300 may be designed (that is, sized and shaped) based upon the patient's actual spina scapulae anatomy. For example, it is common to generate radiographs and/or scans of the shoulder region prior to rTSA, which can be used, such as through computer modeling, to design an implant.

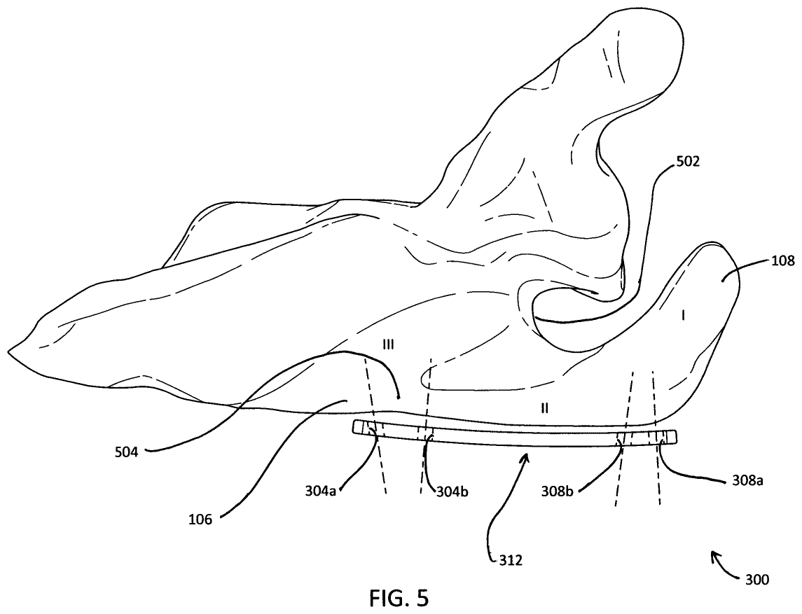

FIG. 4 illustrates an implant 400 similar to that shown in FIG. 3B, except that the implant 400 has a surface reference component 402, which in this embodiment comprises an integral tab that extends away from the concave surface 314 as shown. The tab has an inside surface 404 sized and shaped to contact a superior surface 504 (FIG. 5) of the scapular spine 106 at a medial location. During a minimally invasive procedure, as the implant 400 is inserted through a first lateral incision along the scapular spine 106, the reference component 402 may contact a superior surface of the spine 106 and that contact will locate the implant 400, and more particularly locate the screw head sockets 304a and 304b along the scapular spine 106. It will be appreciated that once the reference component 402 contacts the superior surface the lateral end portion may be articulated or rotated about the reference component 402 as a pivot point to accurately position the lateral end on the scapular spine 106. While the embodiment illustrated in FIG. 4 shows the reference tab 402 at the medial end of the implant 400, it will be appreciated that for some embodiments, a surface reference component 402 may be located in the intermediate portion 312, or in both the medial end portion and the intermediate portion, either continuously or discontinuously.

FIG. 4 also illustrates a spine support component 406 that may be utilized with or without a surface reference component 402. For those fractures caused by downward force or moment applied to the scapular spine, a support component 406 may provide additional stability and strength to avoid or prevent fracture. Although the support component 406 is illustrated as an additional structure emanating from the implant body 302 to contact an inferior surface of the spine, it will be understood that our inventions contemplate the intermediate portion 312 of the body 302 be curved relative to the end portions 306 and 310, such that the end portions contact the posterior edge of the scapular spine 106 and the intermediate portion 312 of the body 302 curves underneath the edge of the spine 106 to contact an inferior surface of the scapular spine. This type of embodiment is preferred when no bone screws will be implanted through the intermediate portion 312.

FIG. 5 illustrates a flat or planar implant 300 positioned along a scapular spine 106 for prophylactic treatment. Medial screw head sockets 304a and 304b are shown positioned in zone III, medial of the spinoglenoid notch 502. The sockets 304a and 304b are shown in diverging orientation such that bone screws (not shown) will enter the scapular spine 106 in non-parallel, diverging orientation to help secure the implant to the spine 106. The intermediate portion 312 of the implant 300 is illustrated to span the majority of zone II and primarily the region between zones II and III about the spinoglenoid notch 502. Lateral screw head sockets 308a and 308b are shown positioned in the lateral portion of zone II at or adjacent to the interface to zone I. The sockets 308a and 308b are shown in non-parallel, converging orientation. Socket 308a is illustrated substantially normal to the implant body 302. Socket 308b is illustrated angled with respect to the implant 300. It will be appreciated that these screw head sockets can all be oriented parallel to the implant, all oriented in converging fashion, all oriented in diverging fashion, or oriented in any combination of angles to the implant body 302.

It is preferred that self-tapping, locking screws, such as 2.7 mm bone screws, are used to secure the implant to the scapular spine 106. Alternately, a combination of self-tapping locking and non-locking bone screws may be used. For example, for a preferred four screw implant, such as implant 300 illustrated in FIG. 5, interior sockets 304b and 308b may be non-locking sockets, such that non-locking bone screws can be employed to first tighten or compress the implant against the edge of the scapular spine 106. Thereafter, locking bone screws can be installed through locking screw sockets 304a and 308a. Additionally, implants according to our inventions may be used with all non-locking bone screws. While it is desirable to use bone screws of all the same size, such as 2.7 mm bone screws, it is contemplated that smaller screws may be used, or screws of differing sizes may be used for a single implant. For example, and not limitation, in the prophylactic example discussed above, the locking screws may be 2.7 mm screws, and the non-locking screws may be of a smaller diameter such as 2 mm or 1.5 mm. It will be appreciated that smaller screws may present less of a stress riser or structural discontinuity, which my help to reduce the possibility of post-rTSA fractures.

While the implant 300 in FIG. 5 is illustrated to be a planar implant, it will be appreciated that any type or shape of implant may be used based on the morphology of the particular scapular spine 106. In this regard, pre-rTSA radiographs or scans of the shoulder joint, including the scapular spine, will benefit selection of the best implant size and shape. With the benefit of this disclosure, those persons of skill will appreciate that scapular spine implants according to our inventions may comprise flat or planar bodies, axially concave or contoured bodies, transversely concave or contoured bodies, axially and transversely concave or contoured bodies, and that each of these body types may further comprise a reference surface, or a support component, or both a reference surface and a support component.

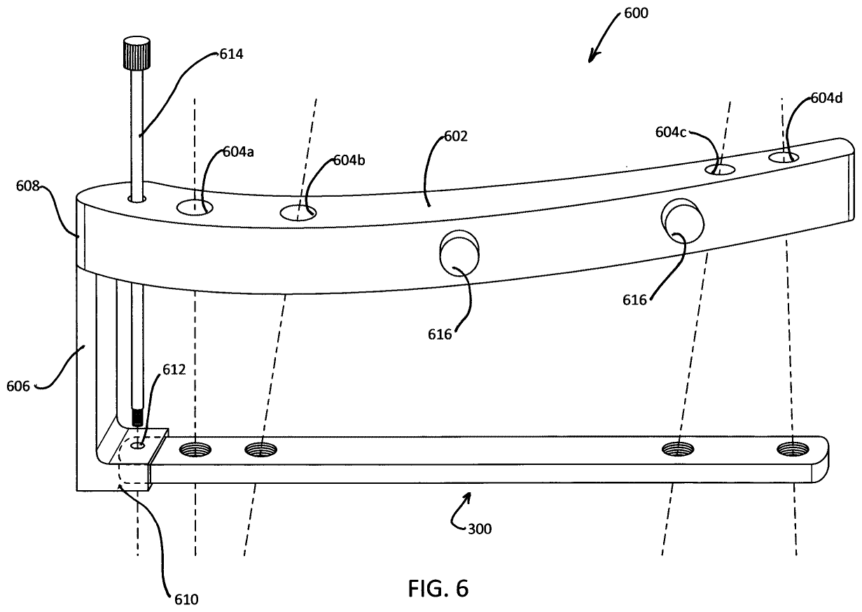

FIG. 6 illustrates an external targeting device 600 that may be used with minimally invasive implantation of scapular spine implants utilizing our inventions. The targeting device 600 comprises an external arm 602 having a plurality of screw guides 604a-d, which correspond to, that is align with, the screw head sockets in the implant. The targeting device 600 also comprises a standoff or leg 606 attached to or integral with the arm 602 at one end 608. The leg 606 comprises an implant receptacle 610 sized and shaped to receive a lateral end of an implant and removable secure the implant to the targeting device. For the embodiment shown in FIG. 6, the receptacle 610 comprises a hole 612 through which an implant attachment screw 614 may be used to removably secure the implant in the receptacle 610. The arm 602 may comprise a plurality of holes or voids 616 to reduce the weight of the arm 602 and/or leg 606.

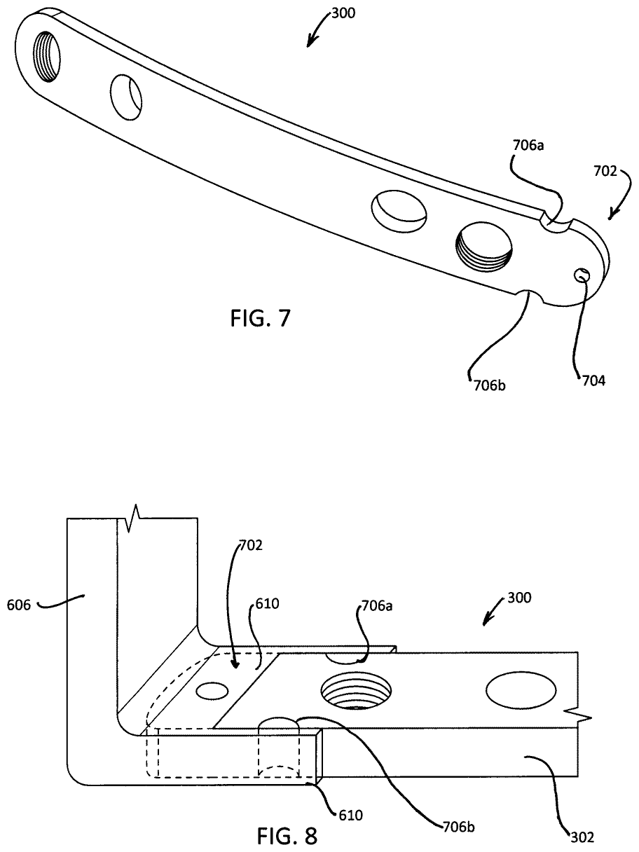

FIG. 7 illustrates an axially concave implant 300 having an integral targeting device portion 702 sized and shaped to mate with the targeting device receptacle 610. A threaded hole 704 is illustrated in the portion 702, which is positioned to align with hole 612 in the receptacle 610. The portion 702 also may comprise locking or aligning surfaces such as grooves or detents 706a and 706b that mate with corresponding tongues in the receptacle. FIG. 8 illustrates how the implant illustrated in FIG. 7 may interface with an external targeting device. The targeting device portion 702 may mate with the receptacle 610 so that the attachment screw 614 secures the implant 300 to the receptacle 610. The combination of the attachment screw 614 and tongue and grooves 706a and 706b secures the implant 300 from movement toward and away from the arm 602 and from side to side during implantation. FIG. 10 illustrates an implant secured to an external targeting device 600.

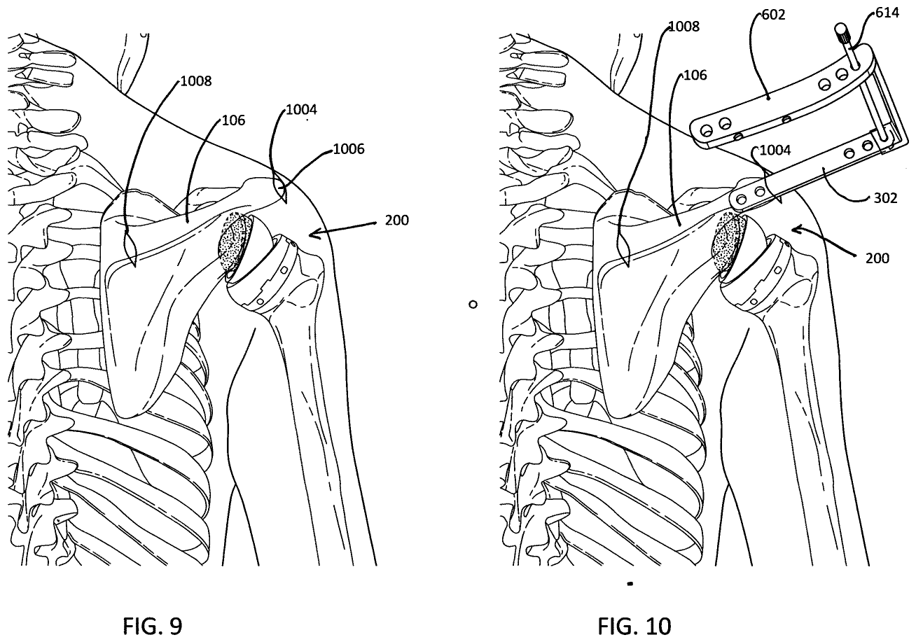

FIG. 9 illustrates the posterior right side of a human shoulder region. The shoulder joint is illustrated as an rTSA prosthesis 200. To implant a scapular spine implant as disclosed herein, it is preferred that an incision 1004 is made adjacent the lateral end 1006 of the acromion. As will be discussed below, also shown is an optional medial incision 1008. FIG. 10 illustrates the combination of an external targeting device 600 and implant 300, being inserted through the lateral incision 1004 and the implant moved along the scapular spine 106 by manipulating the external targeting device 600. For ease of illustration only and not for limitation, the implant is illustrated as planar.

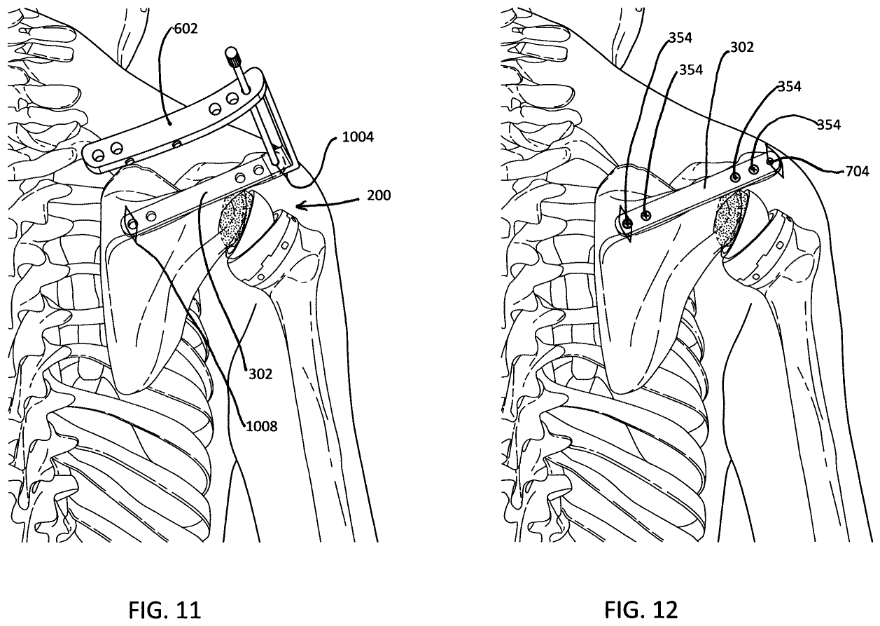

FIG. 11 illustrates the implant 300 fully positioned along the scapular spine 106 prior to bone screw implantation. If the implant comprises a reference surface, the lateral end of the implant/targeting device can be moved, such as rotated relative to the reference surface pivot to accurately locate the lateral end of the implant on the scapular spine. The benefit of the optional medial incision can be appreciated for implants that do not employ a reference surface. For implants that employ a support surface, the external targeting device may be manipulated so that the support surface contacts the underside or inferior surface of the scapular spine during screw implantation. FIG. 12 illustrates an implant, such as implant 300, fully secured to the scapular spine 106 as disclosed herein.

Embodiments of our inventions may have an implant thickness in a range of between about 3 mm and about 10 mm, and preferably in a range of between 4 mm and 7 mm depending on the material from which the implant is made. The implant screw holes may have a diameter in range of between about 1.0 mm and about 4.0 mm, and preferably between about 1.5 mm and 3.0 mm, for use with bone screws of corresponding size.

Other and further embodiments utilizing one or more aspects of the inventions described above can be devised without departing from the spirit of Applicant's invention. Further, the various methods and embodiments of the methods of manufacture and assembly of the system, as well as location specifications, can be included in combination with each other to produce variations of the disclosed methods and embodiments. Discussion of singular elements can include plural elements and vice versa.

The order of steps can occur in a variety of sequences unless otherwise specifically limited. The various steps described herein can be combined with other steps, interlineated with the stated steps, and/or split into multiple steps. Similarly, elements have been described functionally and can be embodied as separate components or can be combined into components that have multiple functions.

The inventions have been described in the context of preferred and other embodiments and not every embodiment of the invention has been described. Obvious modifications and alterations to the described embodiments are available to those of ordinary skill in the art. The disclosed and undisclosed embodiments are not intended to limit or restrict the scope or applicability of the invention conceived of by the Applicants, but rather, in conformity with the patent laws, Applicants intend to protect fully all such modifications and improvements that come within the scope or range of equivalent of the following claims.

1. A prophylactic scapular spine implant used in conjunction with reverse total shoulder arthroplasty, comprising:
an elongated metal plate having an inside surface;
the plate having an end-to-end length sufficient to span from a portion of the acromion to a portion of spina scapulae medial of the spinoglenoid notch;
the plate having a first end portion for associating with the portion of the spina scapulae medial of a spinoglenoid notch, and a second end portion for associating with the portion of the acromion;
the plate having one or more screw head sockets in the first end portion, wherein the one or more sockets are oriented with respect to the plate to create angled screw placement in the spina scapulae;
the plate having one or more screw head sockets in the second end portion, wherein at least one of the sockets is oriented with respect to the plate to create an angled screw placement;
at least one screw head socket in each of the first and second end portions comprising screw locking threads;
the plate having an intermediate portion between the first and second end portions with no screw head sockets therein;
the plate having an implant locating artifact associated with the first end portion and extending outwardly away from the inside surface, the artifact sized and shaped to contact a superior surface of the spina scapulae medial of the spinoglenoid notch and thereby locate the first portion of the plate in relation to the spina scapulae;
wherein during a non-open reduction implantation of the plate, the first end portion of the plate may be inserted through a first incision located adjacent the acromion and the plate slid along the spina scapulae and positioned so that the artifact contacts the superior surface of the spina scapulae medial of the spinoglenoid notch and the intermediate portion spans a portion of the spina scapulae from the acromion to at least the spinoglenoid notch; and
wherein the first portion is secured to the spina scapulae with one or more screws medial to the spinoglenoid notch and the second end portion is secured to the spina scapulae and/or the acromion with one or more screws.

2. The implant of claim 1, wherein the elongated implant is planar.

3. The implant of claim 1, wherein the elongated implant is concave about an axial axis.

4. The implant of claim 1, wherein the elongated implant is concave about a transverse axis.

5. The implant of claim 1, wherein the elongated implant is concave about both an axial axis and a transverse axis.

6. The implant of claim 1, wherein the end-to-end length is between about 7 mm and about 10 mm.

7. The implant of claim 6, wherein first portion comprises two screw head sockets and the second portion comprises two screw head sockets.

8. The implant of claim 7, wherein the screw head sockets in the first and second portions are locking screw sockets.

9. The implant of claim 7, wherein the screw head sockets in the first and second portions are non-locking screw sockets.

10. The implant of claim 7, wherein one of the screw head sockets in the first portion and one of the screw head sockets in the second portion are locking screw sockets.

11. A method of prophylactically implanting the scapular spine implant of claim 1, comprising:
assembling the implant in an external targeting device;
before, during, or after a reverse total shoulder arthroplasty, making a lateral incision adjacent the lateral end of the acromion;
manipulating the external targeting device to introduce a medial end of the implant into the body through the lateral incision;
moving the implant along the scapular spine so that the intermediate portion is adjacent Levy zone II;
contacting the implant locating artifact to a portion of a superior surface of the scapular spine;
adjusting the location of the implant relative to the scapular spine by moving the implant about the implant locating artifact;
implanting bone screws to secure the implant to the scapular spine.

12. A method of stabilizing a scapular spine fracture using the implant of claim 1, comprising:
assembling the implant in an external targeting device;
making a lateral incision adjacent the lateral end of the acromion;
manipulating the external targeting device to introduce a medial end of the implant into the body through the lateral incision;
moving the implant along the fractured scapular spine so that the intermediate portion is adjacent Levy zone II;
contacting the implant locating artifact to a portion of a superior surface of the scapular spine;
adjusting the location of the implant relative to the scapular spine by moving the implant about the implant locating artifact;
implanting bone screws to secure the implant to the scapular spine.
